(19) United States
(12) Patent Application Publication
(10) Pub. No.: US 2025/0114403 A1
(43) Pub. Date: Apr. 10, 2025

(54) USE OF HUMAN MESENCHYMAL STEM CELL SHEET IN TREATMENT OF UTERINE SCARS

(71) Applicants: BOE Regenerative Medicine Technology Co.,Ltd., Beijing (CN); BOE Technology Group Co., Ltd., Beijing (CN)

(72) Inventors: Dehua CHANG, Beijing (CN); Shuang GAO, Beijing (CN); Yufei ZHAO, Beijing (CN); Shuai LIU, Beijing (CN)

(21) Appl. No.: 18/694,962

(22) PCT Filed: Jul. 6, 2023
(86) PCT No.: PCT/CN2023/106157

§ 371 Date: Mar. 23, 2024

(30) Foreign Application Priority Data
Jul. 25, 2022 (CN) 202210877855.7

(51) Int. Cl. A61K 35/28; A61P 15/00

(52) U.S. Cl. CPC A61K 35/28; A61P 15/00

(57) ABSTRACT

A use of a human mesenchymal stem cell sheet in the preparation of a drug for treating uterine scars, and a use of a human mesenchymal stem cell sheet in the preparation of a composition for treating a uterine scar in a subject. The human mesenchymal stem cell sheet is locally applied to the uterus of the subject. The human mesenchymal stem cell sheet can realize site-directed transplantation of mesenchymal stem cells, and no loss would occur because the cells are constrained by extracellular matrixes, such that the cells can directionally release cytokines at a scar site to exert functions.

### CROSS-REFERENCE TO RELATED APPLICATIONS

The present application is a U.S. National Phase Entry of International PCT Application No. PCT/CN2023/106157 having an international filing date of Jul. 6, 2023, which claims priority from Chinese Patent Application No. 202210877855.7 filed on Jul. 25, 2022. The above-identified applications are incorporated by reference herein in their entireties.

### TECHNICAL FIELD

The present disclosure relates to the technical field of stem cell application, and particularly to the use of human mesenchymal stem cell sheet in the treatment of uterine scars.

### BACKGROUND

Endometrium is a highly regenerative reproductive organ. Women will experience more than 400 times of endometrial shedding and regeneration in their whole life. The thickness of endometrium reflects the functional state of endometrium. After endometrial damages such as induced abortion and cesarean section, scars are easy to form and seriously affect embryo nidation of fertilized ovum and pregnancy. Although drugs and manipulations are clinically used to repair the damaged endometrium, the therapeutic effect is not good. In recent years, the in-depth development of stem cell research provides the possibility and prospect for endometrial scar repair, and also brings hope for patients with uterine scar. In recent years, a large number of animal experiments and clinical studies all over the world have shown that mesenchymal stem cells from various tissues can serve to repair and regenerate endometrium, thus treat endometrial scars. Mesenchymal stem cells release angiogenic factors through paracrine, which can reduce local inflammatory reaction of endometrium, improve angiogenesis ability of affected site, reduce the formation of fibrotic scar, and serve to repair and regenerate endometrium. Stem cell therapy often employs local or intravenous injection of stem cell suspension, but will be accompanied by loss of a large number of stem cells in the treatment process, and stem cells cannot be concentrated and collected in the focus to take effect, and due to the low cell utilization rate, the treatment effect is not good. Another method is to use absorbable biomaterials as scaffolds for stem cell transplantation. Although the combination of biological scaffold and cells can solve the problem of cell colonization, the biological scaffold cannot be completely absorbed and residual biological scaffold at affected site will induce local inflammatory reaction and affect the final therapeutic effects.

### SUMMARY

In order to solve the above problems in stem cell transplantation methods, the applicant chose scaffold-free cell sheet technology to improve the transplantation rate of stem cell and the therapeutic effect of endometrial scar repair. Using temperature-sensitive intelligent culture dish, cell sheet technology cultures cells and prepares the cells and their extracellular matrixes into a sheet as a lamellar preparation. Not only mesenchymal stem cell sheet prepared by this technology fully preserves the extracellular matrix secreted by mesenchymal stem cells, but also cells in the sheet are closely connected to each other. Extracellular matrix is an important basic material for mesenchymal stem cells to maintain their physiological functions. Cell sheets with extracellular matrix are not only conducive to the attachment of cell sheets to local tissues, but also contribute to the secretion of cytokines and the transmission of physiological signals between cells, thus making cell sheets play a greater therapeutic role. Cell sheets solves the problem of local colonization of mesenchymal stem cells after transplantation and the problem of how to enhance the curative effect of the stem cells.

The following is a summary of subject matters described herein in detail. The summary is not intended to limit the scope of protection of the claims.

Embodiments of the present disclosure provide the use of a human mesenchymal stem cell sheet in the treatment of uterine scars in a subject, wherein the human mesenchymal stem cell sheet is locally applied to the uterus of the subject.

Embodiments of the present disclosure provide the use of a human mesenchymal stem cell sheet in the preparation of a composition for the treatment of uterine scars in a subject, wherein the human mesenchymal stem cell sheet is locally applied to the uterus of the subject.

Embodiments of the present disclosure provide a method for treating uterine scars in a subject, wherein the method comprises locally applying the human mesenchymal stem cell sheet to the uterus of the subject.

In some exemplary embodiments, the human mesenchymal stem cell sheet may be a semicircle with a diameter of about 3-55 mm semicircular or a suitable shape of approximate area.

In some exemplary embodiments, the human mesenchymal stem cell sheet may be attached to a suture site of the endometrial incision.

In some exemplary embodiments, the diameter of the human mesenchymal stem cell sheet may be a circle with a diameter of 13-20 mm, 22-28 mm, or 35-55 mm, or a semicircle with a diameter of 13-20 mm, 22-28 mm, or 35-55 mm.

In some exemplary embodiments, the human mesenchymal stem cell sheet may be suitably tailored and segmented to make it have other thicknesses or shapes suitable for application.

In some exemplary embodiments, the mesenchymal stem cells of the human mesenchymal stem cell sheet may be derived from umbilical cord, placenta, amniotic sheet, endometrium, adipose tissue, bone marrow, or dental pulp.

In some exemplary embodiments, the human mesenchymal stem cells can be umbilical cord mesenchymal stem cells.

In some exemplary embodiments, the human mesenchymal stem cell sheets may be prepared using mesenchymal stem cells of passage number P0-P20, such as P2-P10.

In some exemplary embodiments, the thickness of the human mesenchymal stem cell sheet can be 10-300 microns, for example, 50-300 microns.

In some exemplary embodiments, the human mesenchymal stem cell sheet can be at a cell density of 1×10⁵ to 1×10⁷/cm², for example, 3×10⁵ to 5×10⁶/cm².

In some exemplary embodiments, the human mesenchymal stem cell sheet may be left standing for 15-60 minutes after attachment so that the human mesenchymal stem cell sheet is completely attached to a tissue of endometrium.

In some exemplary embodiments, after attachment, the human mesenchymal stem cell sheet may be left standing for 15-30 minutes, during which moist sterile gauzes are used to maintain the moisture of the sheet attachment area.

In some exemplary embodiments, the cells in the human mesenchymal stem cell sheets may be connected to each other via their secreted extracellular matrix, which is rich in fibronectin and integrin β1.

In some exemplary embodiments, the human mesenchymal stem cell sheet is capable of secreting a plurality of cytokines, including one or more of HGF, IL-6, IL-8, and VEGF.

The present disclosure has the following advantages:

Human mesenchymal stem cell sheet comprises extracellular matrix secreted by human mesenchymal stem cells, which does not need extra suture and adhesion during transplantation, which is beneficial to the surgical process.

Human mesenchymal stem cell sheet comprises extracellular matrix secreted by human mesenchymal stem cells, compared with the treatment with human mesenchymal stem cell suspension, it can realize site-directed transplantation of mesenchymal stem cells. No loss would occur because the cells are constrained by extracellular matrix, such that the cells can directionally release cytokines at a scar site to exert functions.

Compared with human mesenchymal stem cell suspension, human mesenchymal stem cell sheet has better factor secretion function.

In vivo experimental results in animals show that, compared with the model control group, human mesenchymal stem cell sheet can significantly increase the thickness of myometrium after healing at a uterine injury site, and reduce scar formation (reduction of fibrosis area).

Other aspects of the present disclosure may be comprehended after the drawings and the detailed descriptions are read and understood.

### DESCRIPTION OF DRAWINGS

The drawings are used for providing further understanding of technical schemes of the present disclosure, and constitute a part of the specification. The drawings are used for explaining the technical schemes of the present disclosure together with embodiments of the present disclosure, but do not constitute limitations on the technical schemes of the present disclosure. Shapes and sizes of one or more components in the drawings do not reflect true scales, and are only intended to schematically describe the contents of the present disclosure.

FIG. 1 shows photographs of a model and a cell sheet attached to the model (the cell sheet was indicated by black arrow).

FIG. 2 shows an umbilical cord mesenchymal stem cell sheet.

FIG. 3 shows a cell sheet attached to the scar of uterine incision 0 days after operation (the dashed line area is the cell sheet).

FIG. 4 shows the results of hematoxylin-eosin staining of uterine tissues in experimental group and control group after operation (myometrial thickness was marked by black double arrow).

FIG. 5 shows the results of Masson staining of uterine tissues in experimental group and control group after operation (the blue area indicated by black arrow was the fibrosis area in myometrium).

FIG. 6 shows the statistical results of myometrial thickness and fibrosis of uterine tissue in experimental group and control group (* p<0.05, *** p<0.001).

FIG. 7 shows a comparison of the amount of factors secreted by human umbilical cord mesenchymal stem cell sheet versus human umbilical cord mesenchymal stem cell suspension. It can be seen that, the samples provided by three different donors all show that the human umbilical cord mesenchymal stem cell sheet can secrete more influencing factors.

### DETAILED DESCRIPTION

To make objectives, technical schemes, and advantages of the present disclosure clearer, the embodiments of the present disclosure will be described in detail. It is to be noted that the embodiments in the present disclosure and features in the embodiments may be randomly combined with each other if there is no conflict.

The embodiments of the present disclosure will be described below with reference to the drawings in detail. Implementation modes may be implemented in multiple different forms. Those of ordinary skills in the art may easily understand such a fact that implementations and contents may be transformed into one or more forms without departing from the purpose and scope of the present disclosure. Therefore, the present disclosure should not be explained as being limited to only the contents described in following implementation modes. The embodiments and features in the embodiments of the present disclosure may be randomly combined with each other if there is no conflict.

All technical and scientific terms used herein have the same meanings as understood by those of ordinary skill in the art to which the present disclosure pertains, unless otherwise defined. Although any methods and materials similar or equivalent to those described herein may be used to practice or test the present disclosure, preferred methods and materials are described. For the purposes of the present disclosure the following terms are defined hereinafter.

In this application, unless otherwise explicitly stated, the use of the singular includes the plural. It must be noted that the singular forms “a”, “an” and “the” as used in this specification include plural references, unless the context clearly dictated otherwise. In this application, unless otherwise stated, the use of “or” means “and/or”.

The terms “about” or “approximately” mean to be within an acceptable error range of a particular value, as determined by one of ordinary skill in the art, which will depend in part on how the value is measured or determined, i.e. the limitations of the measurement system. For example, “about” may mean to be within one or more than one standard deviations according to the practice in the art. Alternatively, “about” may mean a range of up to 20%, up to 10%, up to 5%, or up to 1% of a particular value. In other examples, an amount of “about 10” includes 10 and any amount from 9 to 11.

In yet other instances, the term “about” referring to a reference value may also include a range of values of plus or minus 10%, 9%, 8%, 7%, 6%, 5%, 4%, 3%, 2%, or 1% of that value. Alternatively, particularly in relation to biological systems or processes, the term “about” may mean to be within an order of magnitude of a value. When specific values are described in the present application and claims, the term “about” is assumed to mean to be within an acceptable margin of error for the specific values unless otherwise stated.

As use in this specification and one or more claims, the words “comprising” (and any form of comprising, such as “comprise” and “comprises”), “having” (and any form of having, such as “have” and “has”), “including” (and any form of including, such as “include” and “includes”) or “containing” (and any form of containing, such as “contains” and “contain”) are inclusive or open, and do not exclude additional, unlisted elements or method steps. It is contemplated that any of the embodiments discussed in this specification may be implemented with reference to any method or combination of the present disclosure and vice versa. In addition, the combination of the present disclosure may be used to implement the method of the present disclosure.

Human umbilical cord mesenchymal stem cells: mesenchymal stem cells (MSC) are isolated from human umbilical cord Wharton's Jelly. MSC are easy to expand in vitro, express stem cell surface markers, have multi-directional differentiation potential, and secrete cytokines and various growth factors.

Extracellular matrix (ECM): biological macromolecular substances secreted by cells into extracellular stroma. ECM constitutes a complex protein grid structure, which plays a role of supporting, connecting and regulating physiological activities and signaling between cells.

### EXAMPLES

The present disclosure provides preparation of an animal model of endometrial scar. Rabbits (or rats) were selected as experimental animals; a pharmacodynamic evaluation model of mesenchymal stem cell sheet was established; a reproducible, stable and easy-to-popularize uterine incision scar model was developed. The human mesenchymal stem cell sheet of the present disclosure promotes the repair of scar uterine model (reflected by the increase of myometrial thickness and the reduction of fibrosis area after healing)

### Example 1

The culture system comprised a serum-free culture medium and a coating matrix matched with the culture medium.

The serum-free medium can be (1) basic medium+nutritional additives.

The basic medium can be 1640, DMEM, α-MEM, DMEM/F12, F12, etc.

Nutritional additives include multiple of vitamin C, sodium selenate, hydrocortisone, insulin, transferrin, human serum albumin (expressed in plants), progesterone, putrescine, biotin, sodium pyruvate, ethanolamine, carnitine, amino acids, vitamins, glutathione, linoleic acid, linolenic acid, etc.

The serum-free medium also can be (2) commercial serum-free media, including, but not limited to: CTS™ StemPro™ MSC SFM Kit, MesenCult™-ACF Medium, MesenCult™-ACF Plus Medium, MesenCult™-XF Medium, etc.

Coating matrixes matched with the culture medium include but are not limited to collagen, gelatin, fibronectin, adhesin, vitronectin, etc.

This culture system was suitable for the culture, cell sheet preparation and detection of mesenchymal stem cells derived from various tissues, including but not limited to umbilical cord mesenchymal stem cells, placental mesenchymal stem cells, adipose tissue mesenchymal stem cells and bone marrow mesenchymal stem cells. Umbilical cord mesenchymal stem cells were used in this example.

Umbilical cord mesenchymal stem cells were first subject to primary isolation culture and subculture, and the number of cells was expanded. The procedure was as follows:

### 1. Primary Culture of Umbilical Cord Mesenchymal Stem Cells

The umbilical cord of the newborn obtained (from Beijing OASIS International Hospital, donated by pregnant women with informed consent, which has been approved by the Ethics Committee of OASIS International Hospital) was washed with physiological solution having the same osmotic pressure as human body fluid. Then the arteries, veins and adventitia were removed, and Wharton's Jelly was separated, which was cut into small tissue blocks of 0.1-2 mm, and the tissue blocks were evenly plated in a culture container coated with medium, with a spacing of 2-30 mm between the tissue blocks. The culture container was placed in a cell incubator, and after 2-7 days, a proper amount of complete culture medium was added to cover the tissue block. The umbilical cord mesenchymal stem cells crawled out in 8-21 days, and the crawling cells attached to the wall to grow, showing fibroblast-like shape and homogeneous morphology. When the crawling cells grew to 70%-100% confluence, the tissue mass was removed for cell subculture.

### 2. Subculture of Umbilical Cord Mesenchymal Stem Cells

When the cells grew to 70%-100% confluence, the cells were subcultured: the cells were separated from the culture container by a method including but not limited to digestion by trypsin and similar substances, cell scraping, etc. Cells were dispersed in culture medium by methods including but not limited to stirring, vortexing, etc., and the cells were inoculated in a culture container in a density of 500-100000/cm². A proper amount of culture medium was added, and replaced with a proper amount of fresh culture medium every 1-5 days according to the cell growth state. When the cells grew to 70-100% confluence, repeated the subculture operation. The cultured umbilical cord mesenchymal stem cells grew adherently, showing fibroblast shape and homogeneous morphology.

The resulting umbilical cord mesenchymal stem cells were assayed and identified by the following methods:

### 1. Determination of Growth Curve of Umbilical Cord Mesenchymal Stem Cells

The determination methods of growth curve included but were not limited to MTT method, WST method, DNA content detection method, ATP detection method, etc. Here, WST method was taken as an example for further explanation. The umbilical cord mesenchymal stem cells were dispersed, inoculated into the culture well plate at a density according to the reagent instructions, and the solution was changed according to the normal culture conditions. At a fixed time every day for a period of time, the cell activity was detected according to the instructions, and the data of cell activity or quantity was obtained. WST reagent was used to detect the activity of cells daily within 7 days, which included adding WST reagent into a cell plate incubated according to the recommended proportion in the instruction manual. After incubating in the cell incubator for a fixed time, the absorbance value of the liquid in the well of the cell plate at the wavelength of 450 nm was detected by microplate reader or ultraviolet spectrophotometer, which value was positively correlated with the number of cells. From the results, it can be seen that the cell activity in the well increased with the prolongation of culture time, i.e., it can be inferred that the number of cells increased with the prolongation of culture time.

### 2. Identification of Umbilical Cord Mesenchymal Stem Cells

The umbilical cord mesenchymal stem cells can be identified by methods including but not limited to flow cytometry to detect cell surface marker proteins, three-way differentiation of umbilical cord mesenchymal stem cells, PCR to detect cell expression genes, etc. Here, flow cytometry and three-way differentiation were taken as examples for explanation.

### 2.1 Identification of Surface Markers of Umbilical Cord Mesenchymal Stem Cells

Umbilical cord mesenchymal stem cells were dispersed in culture medium, then centrifuged, suspended in serum or a physiological solution containing 1%-20% serum protein and with the same osmotic pressure as human body fluid, and stained with the purchased reagents according to instructions of the purchased reagent for cell surface marker proteins, which include but are not limited to CD73, CD90, CD105, CD34, CD11B, CD19, CD45 and HLA-DR. The phenotypes of CD73, CD90 and CD105 were positive, with a ratio no less than 95%, and the phenotypes of CD34, CD11B, CD19, CD45 and HLA-DR were negative, with a ratio no more than 2%.

### 2.2 Three-Way Differentiation of Umbilical Cord Mesenchymal Stem Cells

Umbilical cord mesenchymal stem cells had the ability to induce differentiation in the direction of bone, cartilage and adipose tissue. The specific operation for detecting osteogenic and adipogenic differentiation was to inoculate cells into a suitable culture vessel according to the proportion indicated in the instructions of the three-way inducing differentiation reagent, and osteogenic and adipogenic induction culture media were add respectively when the cells detected by osteogenic induction grew to 50%-90% confluence and the cells detected by adipogenic induction grew to more than 90% confluence. During chondrogenic induction, a certain number of cells were centrifuged to the bottom of the centrifuge tube, and then the chondrogenic induction culture medium was added. After the cells were pelleted, the cell pellets were made to leave the bottom of the tube to ensure complete contact with the induction culture medium.

Cells were detected when induced and cultured for more than 7 days. Osteogenic induction may include, but is not limited to, alizarin red, anti-hOsteocalcin staining. Adipogenic induction may include, but is not limited to oil red O, anti-mFABP4staining. And chondrogenic induction may include, but is not limited to alcian blue, safranine O, anti-hAggrecan staining.

The preparation procedure of umbilical cord mesenchymal stem cell sheet was as follows

### 1. Coating Temperature-Sensitive Intelligent Petri Dish

Before preparing sheet with a serum-free culture system and mesenchymal stem cells, a layer of matrix which was beneficial to the attachment of mesenchymal stem cells should be coated on the surface of temperature-sensitive intelligent Petri dish in advance, which included but were not limited to collagen, gelatin, fibronectin, vitronectin, laminin, polyornithine, polylysine, etc. The coating matrix was diluted with physiological buffers such as physiological saline and PBS, and then added into a temperature-sensitive intelligent Petri dish, which then was covered with a Petri dish cover. Then, the temperature-sensitive intelligent Petri dish was placed in the incubator at 37° C., saturated humidity and 5% CO₂ for 0.5-48 h for coating.

### 2. Preparation of Umbilical Cord Mesenchymal Stem Cell Sheet

After the coating was completed, the coating matrix was sucked out from the intelligent Petri dish, and the mesenchymal stem cell suspension was added into the temperature-sensitive intelligent Petri dish. In this example, a temperature-sensitive intelligent Petri dish with a diameter of 35 mm was used, and the amount of cells added was 1.8×10⁵-1.8×10⁷ (most preferably 1×10⁷), and the amount of culture solution added was 1-4 ml (most preferably 3 ml). Then, the temperature-sensitive intelligent Petri dish was placed in the incubator at 37° C., saturated humidity and 5% CO₂, and cultured for 2-48 h. After the culture was completed, the temperature-sensitive intelligent Petri dish was moved to an environment of 4-32° C., and without adding any additional reagents or materials, the cells would be separated from the bottom of the temperature-sensitive intelligent culture dish in layers, into cell sheets. The cell sheet was gray-white, with dense structure and smooth and flat surface. It is round with a size of a diameter of 3-30 mm and the thickness was 50-1000 μm. Histological observation of sections showed that the cell sheet comprised multi-layer cell structure and comprised extracellular matrix components secreted by cells.

The function of the formed cell sheet was tested as follows:

### 1. Detection of Factors Secreted by the Cell Sheet by Enzyme-Linked Immunosorbent Assay (ELISA)

The supernatant during the preparation of the cell sheet was detected according to the instructions of the ELISA kit, and the contents of various factors secreted by the cell sheet were determined, and the factors indicated here included but are not limited to interleukin-6 (IL-6), transforming growth factor-β (TGF-β), prostaglandin E2 (PGE2), hepatocyte growth factor (HGF), epidermal growth factor, fibroblast growth factor, platelet-derived growth factor, vascular endothelial growth factor (VEGF), insulin growth factor, stromal cell-derived growth factor-1, tryptophan metabolizing enzyme (indoleamine 2,3-dioxygenase, IDO) and inducible nitric oxide synthase (iNOS), etc. Here, interleukin-6 (IL-6), hepatocyte growth factor (HGF) and vascular endothelial growth factor (VEGF) were used as examples to detect the factors secreted by an umbilical cord mesenchymal stem cell sheet.

### 2. Observation of the Fibronectin in Cell Sheets by Tissue Sections

The obtained cell sheets above were fixed with paraformaldehyde or formalin fixation solution, and then the cell sheets were made into tissue sections with a thickness of 4-10 μm by paraffin section or frozen section methods, and stained to observe the proteins contained in the extracellular matrix of the cell sheets. The proteins include but are not limited to fibronectin, integrin group, vitronectin, etc. Usually, the nuclei were stained with fluorescent dyes such as DAPI or Hoechst 33258 stain at the same time for auxiliary localization. In addition to immunofluorescence methods, staining observation can also be carried out by means including but not limited to immunohistochemical methods. Here, fibronectin and integrin-β1 were taken as examples. Both fibronectin and integrin-β1 were stained with fluorescein-labeled antibody dyes, and the nuclei were stained with DAPI. It can be seen that the prepared cell sheets comprised abundant fibronectin and integrin-β1.

### 3. Observation of the Surface Structure of Cell Sheets by Scanning Electronic Fiberscope

The obtained cell sheets above were fixed in 2.5% glutaraldehyde fixed solution, then dehydrated by gradient alcohol, and then dried in air to obtain dry cell sheets for scanning electron microscope analysis. The dry cell sheets were pasted on the surface of the sample stages with conductive double-sided adhesive tape, and then the surfaces were sprayed with gold by vacuum magnetron sputtering to make the observed sample surface conductive. The sample stages were placed into a scanning electron microscope (Hitachi S-4800 used) for observation.

### 4. The Prepared Sheets Were Temporarily Stored at 4° C.

### Example 2

The steps of making animal model of uterine incision scar were as follows: (1) animal anesthesia was carried out by inhalation or intravenous injection; (2) abdomen was cut to expose uterus after hair-shaving preoperative skin preparation and disinfection; (3) making uterine through and through wound to construct injury model; (4) the uterus was sutured intermittently or continuously; (5) the uterus was returned back to the abdominal cavity; (6), the abdominal cavity was sutured and closed after disinfection.

In the above step (1), the anesthesia method can be gas anesthesia (isoflurane, Lunanbeite Pharmaceutical Co., Ltd.) or intravenous injection anesthesia (pentobarbital sodium, 30 mg/kg dose).

In step (3), when the uterine through and through wound was made, an incision through the serous coat, the myometrium layer and the endometrium layer of the uterus was cut using sharp scalpel; the direction of incision was consistent with the direction of uterine angle, and the length was 1-20 mm.

In step (4), the uterus was sutured. The endometrial layer, myometrium and serous coat of the uterus should be aligned with corresponding layers and sutured layer by layer.

In step (6), the abdominal cavity was closed and sutured. Abdominal muscles should be sutured layer by layer in order to close the abdominal cavity.

### Example 3

Animal model was used to verify the effectiveness of umbilical cord mesenchymal stem cell sheets. The operation was the above step (4). After the model was established, the mesenchymal stem cell sheet was transplanted and attached to the affected site of the serous coat of the uterus (i.e., at the suture site) and maintained for 30-60 minutes. After the cell sheet was completely attached to the surface of the tissue and organ, the above step (5) was continued and the uterus was returned back to the abdominal cavity, and (6) the abdominal cavity was sutured and closed.

The “Mesenchymal Stem Cell Sheet” of the invention was a mesenchymal stem cell sheet obtained by culturing mesenchymal stem cells using a temperature-sensitive Petri dish, the surface of which was a layer of temperature-sensitive polymer material. When the temperature was lowered, the surface tension of the Petri dish was changed, and the contact binding force between the culture dish and the mesenchymal stem cells was reduced, so that the cells formed a film layer from the surface of the Petri dish, which naturally fell off and cell sheets were harvest.

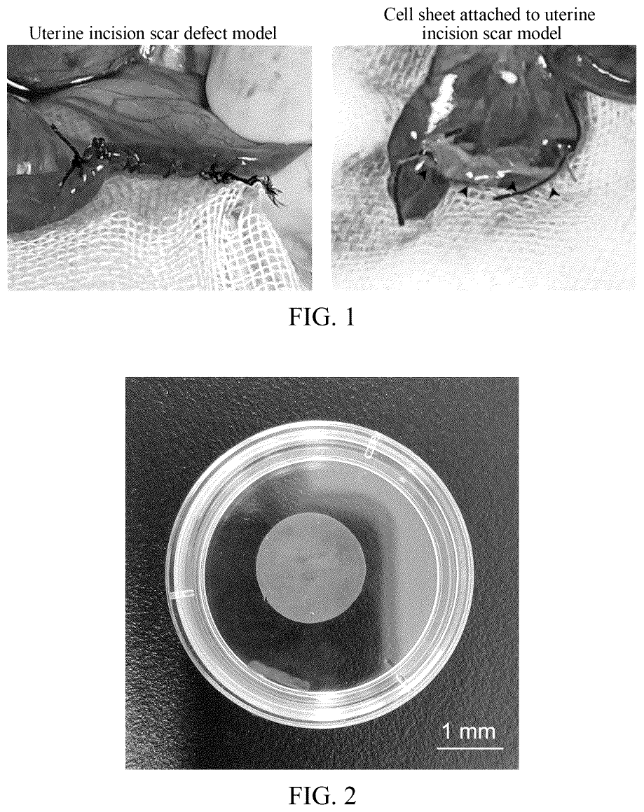

The preparation method of umbilical cord mesenchymal stem cell sheet was as follows: the umbilical cord mesenchymal stem cells were digested into single cell, then plated into a temperature-sensitive Petri dish, the surface of which was linked with polyacrylamide temperature-sensitive polymer materials. The Petri dish was placed in an incubator (Thermo Fisher, Model 371) at 37° C., 5% CO₂ in a 95% humidity environment. After the cells proliferated and were sufficiently confluent, they were moved under an environment at 20° C., the cells would quickly break away from the bottom of the Petri dish in layers, and the umbilical cord mesenchymal stem cell sheets could be harvested (FIG. 2).

The above mesenchymal stem cells could be derived from umbilical cord, placenta, amniotic membrane, endometrium, adipose tissue, bone marrow, dental pulp, etc.

The mechanism of mesenchymal stem cells in the treatment of uterine incision scar was that a variety of growth factors and cytokines, such as HGF, VEGF, IL-8, and the like were secreted after mesenchymal stem cell sheet transplantation. These factors were able to regulate the microenvironment at the site of uterine scar, reduce inflammatory reaction, promote local angiogenesis, reduce fibrosis progress and improve the repair of myometrium of the uterus.

### Example 4

In the method for preparing an animal model of uterine incision scar (experimental rabbit, New Zealand white rabbit, female, weighing 1.8-2.5 kg), the steps were as follow:

(1) an anesthetic was injected intravenously for animal anesthesia;

(2) hair on the rabbit abdomen was removed, and an incision was made in the abdomen to expose uterus after the abdominal skin was disinfected with iodophor (Lircon in Shandong);

(3) the model of uterine through and through wound was made using scalpel, and the wound direction was consistent with the extension direction of uterine angle, the length was 1-20 mm, with a preferable length of 8-15 mm;

(4) the uterus was sutured by using absorbable stylolite (Shanghai Jinhuan, Model 7-0), and the endometrial layer, muscular layer and serous coat should be sutured with each layer being aligned during the suturing process;

(5) the uterus was returned back to the abdominal cavity;

(6) the abdominal muscles were sutured layer by layer with non-absorbable stylolite to close abdominal cavity.

(7) the animals was send to the feeding area and waited to wake up from anesthesia. Only drinking water can be provided within 1 hour after operation, and feed can be provided routinely after stabilization, and kept warm when necessary. Penicillin-streptomycin was continuous oral administrated for three days after operation to prevent postoperative infection.

The animal model was used to verify the effectiveness of a cell sheet of umbilical cord-derived mesenchymal stem cell. After the above step (4), the umbilical cord mesenchymal stem cell sheet was cut to a semicircle with a diameter of about 3-30 mm or an appropriate shape with an approximate area, and attached to the suture site of the endometrial incision. The semicircle with a diameter of 13-20 mm was suitable, which was beneficial to the sheet attachment and was not easy to fall off. The surface of the sheet was flat after attachment (as shown in FIG. 1). The thickness of the mesenchymal stem cell sheet was 10-300 microns, for example, 50-300 microns. The cell proportion of mesenchymal stem cells in mesenchymal stem cell sheet was at least 90%, for example at least 95%. The density of cells in the mesenchymal stem cell sheet was 1×10⁵ to 1×10⁷/cm², for example, 3×10⁵ to 5×10⁶/cm². The mesenchymal stem cell sheets were prepared using mesenchymal stem cells of passage number P0-P20, such as P2-P10.

The umbilical cord mesenchymal stem cell sheet was left standing for 15-60 minutes after attachment, so that the umbilical cord mesenchymal stem cell sheet was completely attached to the endometrial tissue, in which the best time was 15-30 minutes. And moist sterile gauzes were used to maintain the wetting of the sheet attachment area during the standing period.

After that, the above-mentioned operation steps such as (5), (6) and (7) were continued. The organ was returned back to the abdominal cavity. Then the abdominal cavity was closed. After operation, basic nursing such as anti-infection was carried out.

In the process of verifying the effectiveness of umbilical cord mesenchymal stem cell sheet using animal model, the group without cell sheet attached was used as the test control group.

### Example 5

Animal model was used to verify the effectiveness of umbilical cord mesenchymal stem cell sheet. 0 days, 2 weeks and 4 weeks after operation, the uterine samples of experimental and control groups were sampled and fixed, stained with hematoxylin-eosin and Masson. The endometrial tissues of both experimental and control groups were observed under microscope.

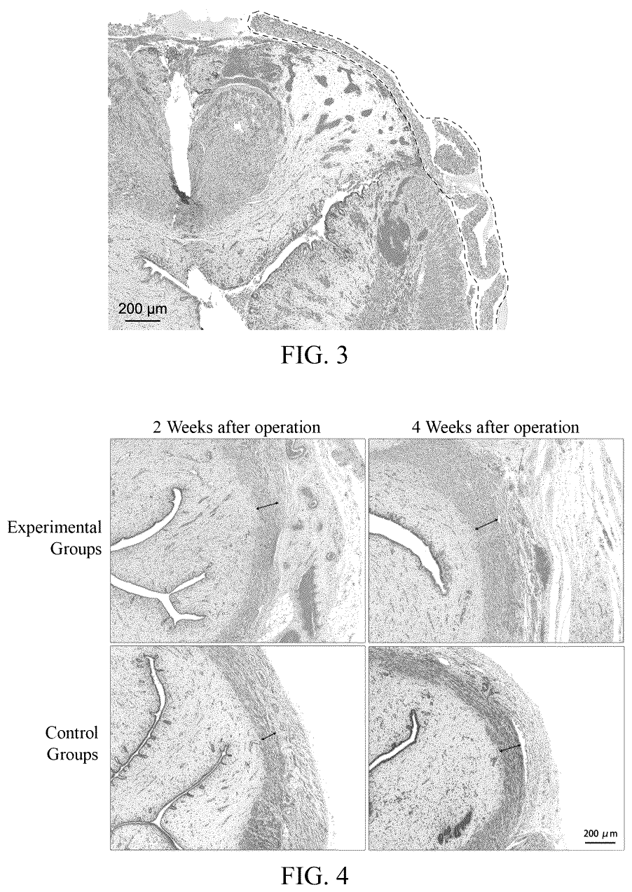

0 days after operation, from the uterine tissue section of the experimental group (hematoxylin-eosin staining (hematoxylin-eosin staining solution, Servicebio)), it can be observed that the cell sheet was attached outside the uterus (shown in FIG. 3).

Two weeks and four weeks after operation, uterine tissue sections (hematoxylin-eosin staining) (FIG. 4) were observed, and the measurement and analysis results of myometrial thickness showed that the experimental group had a therapeutic effect on uterine incision scar after the umbilical cord mesenchymal stem cell sheet transplantation. At different time points after transplantation, the myometrial thickness increased compared with the control group (untreated animals compared with the sheet transplantation group) (the myometrial thickness was shown by black double arrows in FIG. 4). Conclusion: It was showed that the healing time was sped up and the rate of myometrial thickness increasing was improved in the sheet transplantation group (FIG. 6).

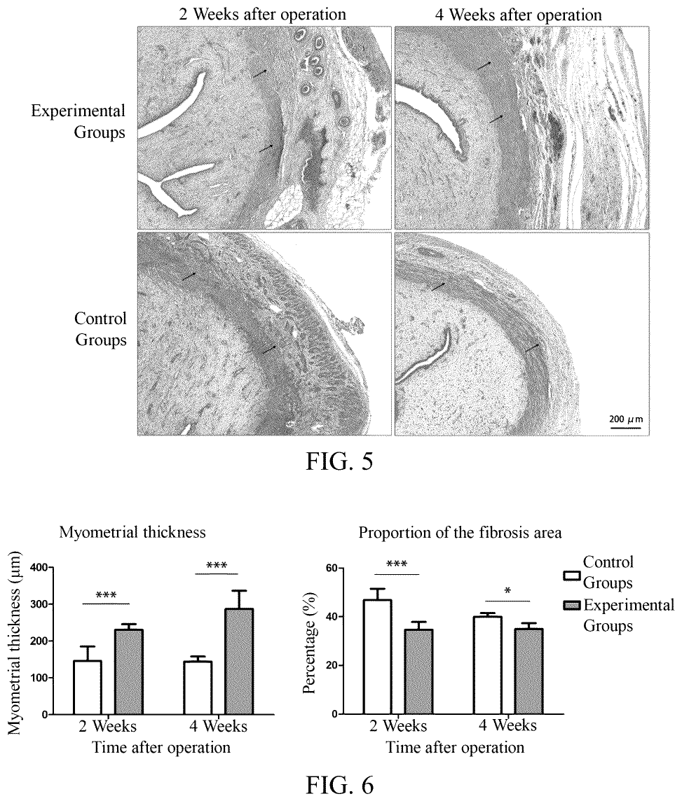

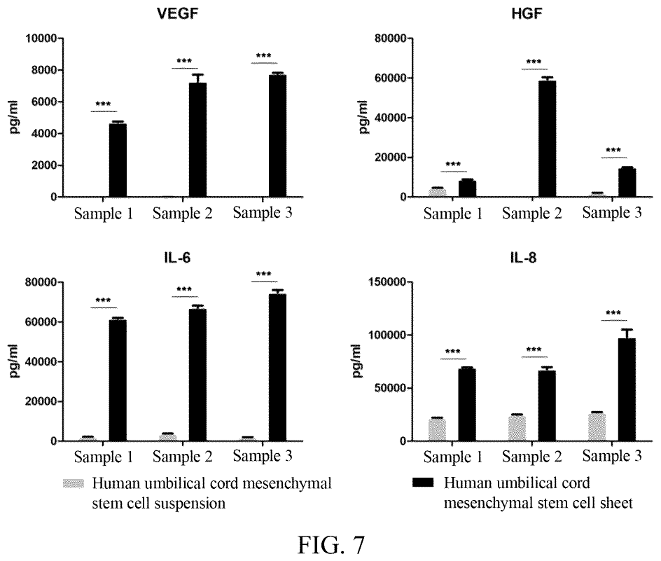

2 weeks and 4 weeks after operation, uterine tissue sections (Masson staining results (Masson trichrome staining solution, Servicebio)) (FIG. 5) were observed, and the measurement and analysis results of the fibrosis area (the blue area indicated by black arrow in FIG. 5 was referred to myometrial fibrosis area) showed that the endometrial fibrosis area in the sheet transplantation group reduced compared with the control group at different time points after operation (FIG. 6).

1. Use of a human mesenchymal stem cell sheet in the treatment of uterine scars in a subject, wherein, the human mesenchymal stem cell sheet is locally applied to the uterus of the subject.

2. Use of a human mesenchymal stem cell sheet in the preparation of a composition for the treatment of a uterine scar in a subject, wherein, the human mesenchymal stem cell sheet is locally applied to the uterus of the subject.

3. A method for treating uterine scar in a subject, wherein, the method comprises locally applying a human mesenchymal stem cell sheet to the uterus of the subject.

4. The use according to claim 1, wherein, the diameter of the human mesenchymal stem cell sheet is a semicircle with a diameter of about 3-55 mm or a suitable shape of approximate area.

5. The use according to claim 1, wherein, the human mesenchymal stem cell sheet is attached to a suture site of endometrial incision.

6. The use according to claim 1, wherein, the human mesenchymal stem cell sheet is a circle with a diameter of 13-20 mm, 22-28 mm, or 35-55 mm, or a semicircle with a diameter of 13-20 mm, 22-28 mm, or 35-55 mm.

7. The use or method according to claim 6, wherein, the human mesenchymal stem cell sheet is suitably tailored and segmented to make it have other thicknesses or shapes suitable for application.

8. The use according to claim 1, wherein, the mesenchymal stem cells of the human mesenchymal stem cell sheet are derived from umbilical cord, placenta, amniotic membrane, endometrium, adipose tissue, bone marrow, or dental pulp.

9. The use according to claim 1, wherein, the human mesenchymal stem cells are umbilical cord mesenchymal stem cells.

10. The use according to claim 1, wherein, the human mesenchymal stem cell sheets are prepared using mesenchymal stem cells of passage number P0-P20, such as P2-P10.

11. The use according to claim 1, wherein, the thickness of the human mesenchymal stem cell sheet is 10-300 microns, for example, 50-300 microns.

12. The use according to claim 1, wherein, the human mesenchymal stem cell sheet is at a cell density of 1×10⁵ to 1×10⁷/cm², for example, 3×10⁵ to 5×10⁶/cm².

13. The use according to claim 1, wherein, the human mesenchymal stem cell sheet is left standing for 15-60 minutes after attachment so that the human mesenchymal stem cell sheet is completely attached to a tissue of endometrium.

14. The use according to claim 1, wherein, after attachment, the human mesenchymal stem cell sheet is left standing for 15-30 minutes, during which moist sterile gauzes are used to maintain the moisture of sheet attachment area.

15. The use according to claim 1, wherein, the cells in the human mesenchymal stem cell sheets are connected to each other via their secreted extracellular matrix, which is rich in fibronectin and integrin β1.

16. The use according to claim 1, wherein, the human mesenchymal stem cell sheet is capable of secreting a plurality of cytokines, including one or more of HGF, IL-6, IL-8 and VEGF.

17. The use according to claim 2, wherein, the diameter of the human mesenchymal stem cell sheet is a semicircle with a diameter of about 3-55 mm or a suitable shape of approximate area.

18. The method according to claim 3, wherein, the diameter of the human mesenchymal stem cell sheet is a semicircle with a diameter of about 3-55 mm or a suitable shape of approximate area.

19. The method according to claim 2, wherein, the human mesenchymal stem cell sheet is a circle with a diameter of 13-20 mm, 22-28 mm, or 35-55 mm, or a semicircle with a diameter of 13-20 mm, 22-28 mm, or 35-55 mm.

20. The use according to claim 3, wherein, the human mesenchymal stem cell sheet is a circle with a diameter of 13-20 mm, 22-28 mm, or 35-55 mm, or a semicircle with a diameter of 13-20 mm, 22-28 mm, or 35-55 mm.
